(19) United States
(12) Patent Application Publication
(10) Pub. No.: US 2025/0311955 A1
(43) Pub. Date: Oct. 9, 2025

(54) SMART TERMINAL FOR ACQUIRING MEDICAL INFORMATION INCLUDING AUDITORY ASPECT

(71) Applicant: SEOUL NATIONAL UNIVERSITY HOSPITAL, Seoul (KR)

(72) Inventor: Joonghee KIM, Seongnam-si, Gyeonggi-do (KR)

(21) Appl. No.: 18/856,565

(22) PCT Filed: Apr. 13, 2023
(86) PCT No.: PCT/KR2023/005025

§ 371 Date: Jun. 20, 2025

(30) Foreign Application Priority Data
Apr. 15, 2022 (KR) 10-2022-0047132

(51) Int. Cl. A61B 5/346; A61B 5/00

(52) U.S. Cl. CPC A61B 5/346; A61B 5/6823; A61B 5/7264

(57) ABSTRACT

Embodiments relate to a smart terminal for acquiring medical information including an auditory aspects, comprising one or more processors and a memory for storing instructions executed by the one or more processors, wherein the instructions, when executed by the one or more processors, allow the one or more processors to cause: at least one acoustic unit connected to the body of the smart terminal to acquire body sound of a subject; a data processing module included in the smart terminal to process, in a preset data format, raw body information about body sound and/or a body electrocardiogram, so as to generate medical information about the subject; and a pressure switch included in the smart terminal to generate an electrical signal when switched by the pressure applied thereto.

### TECHNICAL FIELD

The present application relates to a technology of acquiring medical information in real time, and more particularly, to a smart terminal that acquires medical information in real time in a format that may be utilized for clinical treatment only by using in a medical process.

### BACKGROUND ART

A physician's fast and accurate information processing capability is required for successful medical treatment. In general clinical treatment, examination and auscultation treatments account for a large proportion. Therefore, it is important that medical information acquired in the examination and auscultation treatment processes is recorded in a form (or format) that may be quickly used.

However, conventional equipment that is widely used at present in most examination and auscultation treatments are smart phones, digital cameras, recorders, and the like, and there is a possibility that no record may be left in the auscultation treatment process.

In particular, the equipment is unsuitable for the automation of clinical treatment in recent years, as they have been designed to be non-suitable for medical information formats for clinical treatment. Various diagnostic programs are being utilized in clinical treatment, and in recent years, attempts have been made to incorporate artificial intelligence algorithms into these diagnostic programs.

Conventional equipment has limitations in storing a non-suitable form of raw body information that cannot be immediately utilized in a diagnostic program or an artificial intelligence program even if records of the auscultation, and examination processes are left.

### DETAILED DESCRIPTION OF THE INVENTION

### Technical Problem

The present application may provide, in one aspect, a smart terminal including at least an auditory aspect, and further capable of acquiring medical information of an electrocardiogram aspect and/or medical information of a visual aspect.

In addition, in another aspect, this application may provide a smart terminal capable of acquiring medical information of an auditory aspect or a visual aspect.

### Technical Solution

A smart terminal for acquiring medical information including an auditory aspect, comprises: one or more processors and a memory for storing instructions executed by the one or more processors, wherein the instructions, when executed by the one or more processors, allow the one or more processors to: cause at least one acoustic unit connected to the body of the smart terminal to acquire body sound of a subject, and cause a data processing module included in the smart terminal to process at least one raw body information among the acquired body sound and body electrocardiogram into a preset data format to generate medical information of the subject, and a pressure switch included in the smart terminal generates an electric signal when pressure is applied and and switched.

In one embodiment, the instructions, when executed by the one or more processors, allow the one or more processors to: cause the acoustic unit to initiate a listening operation in response to the electric signal of the pressure switch being generated.

In one embodiment, the interfaces of at least one acoustic unit are arranged on the same plane to form a unit array, and the unit array is connected to a first coupling portion of the body, and the first coupling portion protrudes more than a surface of another portion of the body.

In one embodiment, the pressure switch is arranged between the first coupling portion and the unit array, the instructions, when executed by the one or more processors, allow the one or more processors to: apply pressure to the pressure switch and generate the electrical signal according to the contact of the protruding unit array with the surface of the body part from which body sound is to be acquired.

In one embodiment, it further comprises: a 2D cross-sectional ultrasonic sensor or an ultrasonic sensor using the Doppler effect.

In one embodiment, the instructions, when executed by the one or more processors, allow the one or more processors to: cause the plurality of acoustic units included in the unit array to listen to body sound of different frequency bands, cause a first acoustic unit included in the plurality of acoustic units to listen to relatively high-pitched body sound in audible frequency band, and cause a second acoustic unit included in the plurality of acoustic units to listen to relatively low-pitched body sound in the audible frequency band.

In one embodiment, the instructions, when executed by the one or more processors, allow the one or more processors to: cause an operating unit included in the smart terminal to mix the heard high-pitched sound or low-pitched sound, or control the mixing ratio.

In one embodiment, the instructions, when executed by the one or more processors, allow the one or more processors to: cause a sound output unit included in the smart terminal to output the acquired body sound as an sound signal, and cause a display unit included in the smart terminal to display raw body information including body sound acquired, or medical information of a subject.

In one embodiment, the instructions, when executed by the one or more processors, allow the one or more processors to: cause a data transmission/reception unit included in the smart terminal to transmit the medical information of the subject to an external device having a diagnostic program or an artificial intelligence program installed.

In one embodiment, the instructions, when executed by the one or more processors, allow the one or more processors to: cause the data processing module to process the body sound signal into a preset data format to generate medical information of the subject, and the preset data format is a data format acceptable to an application program installed in an external device communicating with the data transmission/reception unit.

In one embodiment, the instructions, when executed by the one or more processors, allow the one or more processors to: cause the data processing module to extract a one-dimensional sound vector x of a preset length N from the body sound, and generate medical information of the subject using the one-dimensional sound vector x itself, or generate medical information of the subject by converting the one-dimensional sound vector x into an image format.

In one embodiment, the instructions, when executed by the one or more processors, allow the one or more processors to: cause the data processing module to convert the one-dimensional sound vector

x into a spectrum format, a power spectrum format, or a spectrogram format to generate sound data in an image format, expressed as a C×M matrix, the medical information of the subject includes the sound data in the image format, and the C represents a channel of body sound, and the M represents time of body sound.

In one embodiment, the instructions, when executed by the one or more processors, allow the one or more processors to: cause at least one photographing unit included in the smart terminal to photograph the body to acquire more medical information of visual aspect, and cause the data processing module to process the body image obtained by the at least one photographing unit into a preset data format so that the medical information of the subject further includes a result of processing the body image.

In one embodiment, the instructions, when executed by the one or more processors, allow the one or more processors to: cause the operating unit included in the smart terminal to select the photographing unit or control the specifications of the photographing unit.

In one embodiment, the at least one photographing unit includes one or more of at least one visible light photographing unit; at least one infrared photographing unit; and at least one illuminance sensor.

In one embodiment, the smart terminal further includes one or more of a white light source and a fluorescent light source when the smart terminal includes at least one visible light photographing unit.

In one embodiment, when the smart terminal includes at least one infrared photographing unit, the instructions, when executed by the one or more processors, allow the one or more processors to: cause the data processing module to generate a temperature image of a photographed area to determine whether there is an inflammatory reaction in a specific area.

### Advantageous Effects

A smart terminal according to embodiments of the present invention may acquire various body sounds, various body images, and body electrocardiograms to generate medical information of a subject of fusion data to be usefully applied to an artificial intelligence algorithm using sensor fusion.

In particular, the smart terminal may easily and effectively perform various tasks related to clinical treatment (photography, auscultation, recording, etc.) while interacting with a patient during clinical treatment, thereby enabling medical professionals to easily and effectively record clinical information acquired in real time.

Furthermore, the medical information of the subject of the fusion data has a specific data format that may be used more effectively by the artificial intelligence program, such as multimodal input data. That is, the smart terminal generates and provides medical information of a subject of multimodal fusion data that may be directly utilized in various artificial intelligence algorithms from raw body information, thereby improving medical efficiency based on artificial intelligence.

The effects of the present invention are not limited to the effects mentioned above, and other effects not mentioned will be clearly understood by those skilled in the art from the description of the claims.

### BRIEF DESCRIPTION OF THE DRAWINGS

In order to describe the technical solutions of the embodiments of the present invention or the prior art more clearly, the drawings required in the description of the embodiments are briefly introduced below. It is to be understood that the following drawings are for the purpose of describing embodiments of the present disclosure only and are not intended to be limiting. In addition, for clarity of description, some elements to which various modifications, such as exaggeration, omission, etc., have been applied may be illustrated in the following drawings.

FIG. 1 is a schematic diagram of a smart terminal according to an embodiment of the present application.

FIGS. 2A and 2B illustrate a unit array for performing an sound listening operation and an electrocardiogram measurement operation, according to an embodiment of the present application.

FIG. 3 illustrates a unit array in which an arrangement of sound units is modified, according to another embodiment of the present application.

FIG. 4 illustrates a process of measuring an electrocardiogram of a subject through a plurality of electrocardiogram units, according to an embodiment of the present application.

FIG. 5 is a diagram illustrating a unit array including a plurality of photographing units, according to an embodiment of the present application.

FIG. 6 illustrates an sound segment partitioned using a measured body electrocardiogram, according to an embodiment of the present application.

FIG. 7 shows a result of synchronizing body electrocardiogram and body sound based on partitioning of the heart cycle, according to an embodiment of the present application.

### DETAILED DESCRIPTION OF THE EMBODIMENTS

The terminology used herein is for the purpose of referring to particular embodiments only and is not intended to be limiting of the invention. As used herein, the singular forms “a,” “an,” and “the” include plural referents unless the phrases clearly indicate the contrary. The meaning of “comprising,” as used herein, embodies a particular property, region, integer, step, operation, element, and/or component and does not preclude the presence or addition of other properties, regions, integers, steps, operations, elements, and/or components.

Although not otherwise defined, all terms, including technical and scientific terms, used herein have the same meaning as commonly understood by one of ordinary skill in the art to which this invention belongs. Terms defined in commonly used dictionaries are further interpreted as having a meaning consistent with the relevant technical literature and the presently disclosed subject matter, and are not to be interpreted in an idealized or very formal sense unless defined.

Hereinafter, embodiments of the present invention will be described in detail with reference to the drawings.

A smart terminal 1 according to the embodiments of this application includes: an auditory input module for acquiring medical information of an auditory aspect; an electrocardiogram input module for acquiring medical information of the electrocardiogram aspect; and/or a visual input module for acquiring medical information of the visual aspect.

In certain embodiments according to an aspect of this application, the smart terminal 1 may acquire medical information of an auditory aspect and medical information of an electrocardiogram aspect. Further, in alternative embodiments, this smart terminal 1 may be further configured to acquire medical information of the visual aspect.

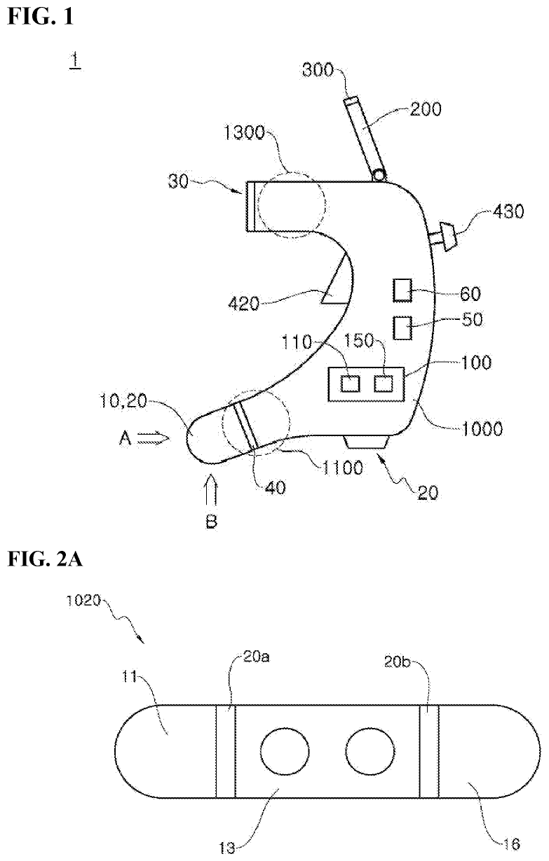

FIG. 1 is a schematic diagram of a smart terminal according to an embodiment of the present application.

Referring to FIG. 1, a smart terminal 1 includes at least one acoustic unit 10; a plurality of electrocardiogram units 20; a data processing module 100; and a body 1000. The body 1000 includes a pressure switch 40 in contact with the electrocardiogram unit 20. Further, in the alternative embodiments, the smart terminal 1 may further include at least one photographing unit 30.

Here, the one acoustic unit 10, the plurality of electrocardiogram units 20, the photographing unit 30, the pressure switch 40, the data processing module 100 and the body 1000 may be implemented or realized by one or more processors included in a smart terminal 1.

In some embodiments, the smart terminal 1 may further include an auxiliary acoustic unit 50, an auxiliary sensor 60, a display unit 200, an audio output unit 300, and/or an operating unit 400.

Here, the auxiliary acoustic unit 50, the auxiliary sensor 60, the display unit 200, the audio output unit 300, and the operating unit 400 may be implemented or realized by one or more processors included in a smart terminal 1.

The acoustic unit 10, the electrocardiogram unit 20, and the photographing unit 30 are input units that interact with the body of the subject to acquire raw body information of the subject.

The auxiliary acoustic unit 50, the auxiliary sensor 60, the display unit 200, the audio output unit 300, and the operating unit 400 are input/output units that interact with a user to generate medical information of a subject.

The smart terminal 1 according to embodiments may have an aspect that is entirely hardware, entirely software, or partly hardware and partly software. For example, an apparatus or a device may collectively refer to hardware with data processing capability and operating software for driving the hardware. In this specification, the terms “unit,” “module,” “device,” “system,” and the like are intended to refer to a combination of hardware and software driven by the hardware. For example, the hardware may be a data processing device including a CPU (Central Processing Unit), a GPU (Graphic Processing Unit), or another processor. In addition, software may refer to an executing process, an object, an executable, a thread of execution, a program, or the like.

The body 1000 includes one or more components (e.g., the data processing module 100) therein. Some other components contained therein are then protected from external material, impact, by the body 1000.

In addition, the body 1000 has a structure in which the units 10, 20, and 30 described above are installed so as to be able to come into contact with the body.

In certain embodiments, the body 1000 may include a first coupling portion 1100. The first coupling portion 1100 may be a portion of the body 1000 connected with the acoustic unit 10 and/or the at least one electrocardiogram unit 20.

In addition, the body 1000 may further include a second coupling portion 1300. The second coupling portion 1300 may be a portion coupled with the photographing unit 30. The first coupling portion 1100 may be a single piece of body 1000, and the second coupling portion 1300 may be another piece of body 1000. For example, as shown in FIG. 1, the first coupling portion 1100 may be a single formed at the bottom of the body 1000. The second coupling portion 1300 may be another single formed on top of the body 1000.

In one embodiment, the first coupling portion 1100 and/or the second coupling portion 1300 may protrude from the other portion of the body 1000 so as to first contact the body relative to the other portion of body 1000. Then, the body 1000 may have a shape in which the thickness of the intermediate portion between the upper portion and the lower portion is relatively narrow. The degree of protrusion of the first coupling portion 1100 and the second coupling portion 1300 may be the same or different from each other. For example, the first coupling portion 1100 and the second coupling portion 1300 protrude as compared to other portions of the body 1000, and the extent of the protrusion may be similar as shown in FIG. 1.

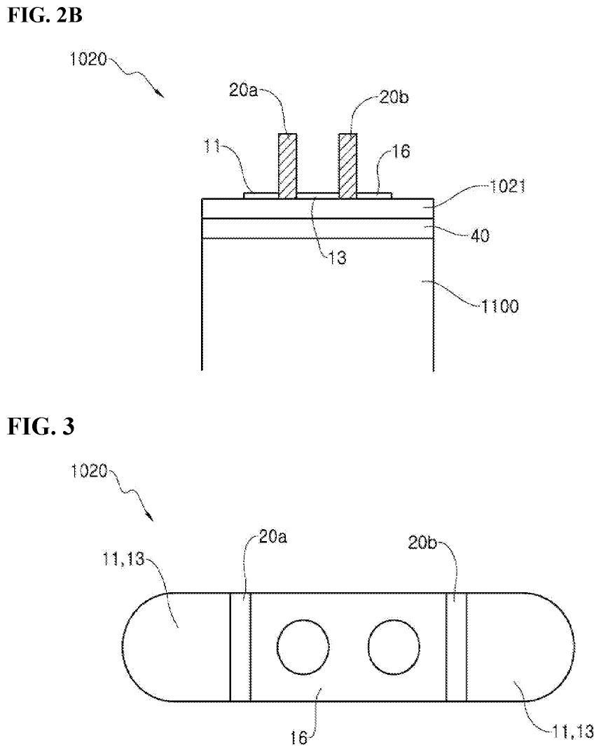

As the first coupling portion 1100 and the second coupling portion 1300 protrude relatively, the body 1000 may have a shape suitable for gripping by a user's hand. In this case, the user may grip a relatively narrow portion between the first coupling portion 1100 and the second coupling portion 1300. As such, the body 1000 provides a convenient shape for use. In one embodiment, some components coupled to the body 1000 may include components coupled such that a distance from the body 1000 is temporarily changed. This will be described in more detail with reference to FIG. 2 below.

The acoustic unit 10 is an input component that acquires body sound. The acoustic unit 10 may directly acquire an audio signal inside the bottom body outside the skin, or may detect vibration inside the body by contacting the skin to acquire an audio signal. The acoustic unit 10 may be, for example, a microphone or a vibration sensor.

The smart terminal 1 may include a plurality of types of acoustic units 10. In the plurality of types of acoustic units 10, some types of the acoustic units 10 and some other types of the acoustic unit 10 are configured to listen to sounds in different frequency bands.

The plurality of types of acoustic units 10 may include a first type of acoustic unit 11 and/or a second type of acoustic unit 13 for listening to sound in an audible frequency band.

The first acoustic unit 11 is an input component that listens to relatively high-pitched body sound in an audible frequency band.

The second acoustic unit 13 is an input component that listens to relatively low-pitched body sound in an audible frequency band.

Then, the smart terminal 1 may generate auditory medical information including information related to the listening low-pitched body sound and/or the high-pitched body sound.

The acoustic unit 11 of the first type and the acoustic unit 13 of the second type may be contact microphones. The part of the contact microphone 11, 13 in contact with the surface of the body may be made of a material having elasticity, such as silicone rubber.

In addition, the acoustic unit 11 of the first type and the acoustic unit 13 of the second type may further include an amplifying unit. The amplification unit may be a component that amplifies physical energy input via a portion in contact with the surface of the body. The amplification unit may be a piezoelectric microphone unit.

In addition, the plurality of types of acoustic units 10 may further include an ultrasonic unit 16. The smart terminal 1 may include one or more ultrasonic units 16. Then, the smart terminal 1 may generate auditory medical information including information related to the heard ultrasonic sound.

The ultrasonic unit 16 listens to body sounds having ultrasonic waves outside the audible frequency. The ultrasonic unit 16 may comprise, for example, a 2D cross-sectional ultrasonic unit and/or a Doppler sensor.

The 2D cross-sectional ultrasound unit 16 has a probe and is an ultrasound sensor that listens to ultrasound in a two-dimensional cross-section of the skin.

The Doppler sensor 16 is an ultrasonic unit that uses a Doppler effect. The Doppler sensor listens to changes in the frequency of ultrasound waves that impinge upon and return to the organ tissue or bloodstream that is directed to or away from the probe.

Further, the ultrasound unit 16 may be utilized to perform lung ultrasound examination. Further, when the ultrasonic sensor 16 is brought into contact with the echocardiographic layer position, the smart terminal 1 may serve as a small echocardiographic device in clinical practice. The electrocardiogram unit 20 is an input component that acquires a body electrocardiogram signal. The electrocardiogram unit 20 may be implemented as an electrode made of a conductive material to receive an electrical signal, such as an electrocardiogram signal.

One or more electrodes (e.g., 20A, 20B) of the plurality of electrocardiogram units 20 contact a heart region of the body. The cardiac region refers to a skin region that is measurable for electrical signals of the heart.

Another electrode (e.g., 20C) of the plurality of electrocardiogram units 20 may be in contact with a region other than the heart region of the body. Then, the smart terminal 1 acquires a weak electric (i.e., potential difference) signal that is measurable on a body surface with a body electrocardiogram through a plurality of electrocardiogram units 20 (e.g., electrodes). The smart terminal 1 is configured to more conveniently listen to the body sound of the subject. In addition, the smart terminal 1 may be configured to be able to measure the body electrocardiogram more accurately and conveniently at the same time as listening to the sound of the body.

FIGS. 2A and 2B illustrate a unit array for performing an sound listening operation and an electrocardiogram measurement operation, according to an embodiment of the present application.

FIG. 2A is a view of a unit array as viewed by arrow a in FIG. 1, and FIG. 3B is a view of a unit array as viewed from arrow b in FIG. 1.

Referring to FIGS. 2A and 2B, the at least one acoustic unit 10 and some electrocardiogram units 20 form a unit array 1020. For example, as shown in FIGS. 2A and 2B, the unit array 1020 may be formed by arranging the acoustic units 11, 13, 16 and the electrocardiogram units 20a, 20b on the same contact surface.

In one embodiment, the unit array 1020 may include a board 1021. At least one acoustic unit 10 and some electrocardiogram units 20 are provided on the board 1021.

The unit array 1020 is configured such that the units 10, 20 are arranged on the contact surface such that an audio listening operation for acquiring body sound and an electrocardiogram measuring operation for measuring body electrocardiograms are simultaneously performed through the contact surface opposite the body.

In one embodiment, the electrocardiogram unit 20 may be configured such that the surface of the electrode in contact with the body forms a higher step than the contact surface of the unit array 1020 and the surface of the surrounding acoustic unit 10. When the unit array 1020 configured with the stepped cross-sectional structure of FIG. 2B contacts the body, the electrocardiogram unit 20 contacts the body before the acoustic unit 10 on or around the contact surface of the unit array 1020. The electrocardiogram signal must propagate through the conductive medium to be accurately measurable. Therefore, in order to more accurately measure the body electrocardiogram, the electrocardiogram unit 20 must stably contact the surface of the body (e.g., the skin of the subject patient). An incorrect body electrocardiogram may be acquired if the electrode of the electrocardiogram unit 20 falls without contacting the surface of the body. On the other hand, the acoustic unit 10 is capable of propagating through a medium such as air, and thus does not necessarily have to be in direct contact with the body. Consequently, when the acoustic unit 10 and the electrocardiogram unit 20 are arranged in the cross-sectional structure of FIG. 2B, the smart terminal 1 may acquire both the body electrocardiogram and the body sound with a relatively small error.

In one embodiment, when the smart terminal 1 includes a plurality of types of acoustic units 10, the plurality of types of the acoustic units 10 and some electrocardiogram units 20a and 20b may be intersectingly arranged in a cross-sectional manner.

For example, the electrocardiogram units 20a, 20b may be arranged between the low-pitched acoustic unit 11; the high-pitched acoustic unit 13; and the ultrasonic unit 16, respectively.

In one embodiment, the acoustic unit 11 of the first type and the acoustic unit 13 of the second type may be provided in the same arrangement region. For example, the acoustic unit 11 of the first type and the acoustic unit 13 of the second type may be installed in a single arrangement region on the left or right side of FIG. 2A, which is divided by the electrocardiogram unit 20a or 20b.

Here, the first acoustic unit 11 and the second acoustic unit 13 may be implemented or realized by one or more processors included in a smart terminal 1.

FIG. 3 illustrates a unit array in which an arrangement of sound units is modified, according to another embodiment of the present application.

Referring to FIG. 3, the arrangement order of the plurality of types of acoustic units 11, 13, 16 of FIG. 2 may be variously modified. The ultrasound unit 16 may be arranged between the electrocardiogram units 20a, 20b as shown in FIG. 3.

Here, the ultrasound unit 16 may be implemented or realized by one or more processors included in a smart terminal 1.

In addition, under such a unit array 1020 structure, the smart terminal 1 may perform lung ultrasound inspection, which listens to lung ultrasound through the ultrasound unit 16, simultaneously with the lung sound auscultation operation, listening to lung sound through the acoustic unit 11 of the first type and the acoustic unit 13 of the second type. This is because the heart is adjacent to the lungs, so that when the unit array 1020 is brought into contact with the heart region, lung sound and/or lung ultrasound may be simultaneously heard through the acoustic unit 10 of the unit array 1020).

In some embodiments, the surface in contact with the body in the unit array 1020 may be made of a conductive material in part or in whole. Then, the area of the conductive region capable of receiving the electrocardiogram signal increases, and the signal reception performance is further enhanced.

In some embodiments, the electrocardiogram unit 20 extending through the connecting member 1200 of FIG. 1 may include a sidewall at least partially made of a non-conductive material. The side wall is a portion touched by a finger of a user. This sidewall portion is treated with a non-conductive material to further suppress the influence of external noise on the electrocardiogram signal acquisition.

In addition, the unit array 1020 is connected to a protruding portion of the body 1000 of FIG. 1. In one embodiment, the unit array 1020 may be connected with the first coupling portion 1100.

When the unit array 1020 is connected to a protruding portion of the body 1000, only the electrocardiogram units 20a, 20b and the acoustic unit 10 of the unit array 1020 may be in contact with the body instead of the other portions of the body 1000. As a result, the user may acquire body sound and body electrocardiogram by moving the smart terminal 1 minimally toward the subject. In addition, a portion where the smart terminal 1 is in contact with the body is minimized, so that discomfort felt by a subject such as a patient is minimized.

In addition, the smart terminal 1 may be more easily configured to initiate an audio listening operation and an electrocardiogram measurement operation.

Referring again to FIG. 1, the smart terminal 1 includes a pressure switch 40 disposed between the unit array 1020 and the body 1000. The unit array 1020 is coupled with the body 1000 (e.g., the first coupling portion 1100) via a pressure switch 40.

The pressure switch 40 is a switching element that generates an electrical signal when a pressure is applied to the switching element to be in an on-state. Since the electrical signal indicates activation of the switch 40, it may also be referred to as an activation signal. The electrical signal generated by the pressure switch 40 may also be transmitted to the data processing module 100. The data processing module 100 may transmit a control signal to another component (e.g., unit 10 or 20) in response to the activation signal.

In one embodiment, the acoustic unit 10 may initiate a listening operation in response to an electrical signal of the pressure switch 40 being generated. The electrical signal of the pressure switch 40 is a signal generated when the switch is turned on, and is generated when the combined unit array 1020 contacts the body and pressure is applied.

The electrical signal of the pressure switch 40 is a trigger signal for initiating an sound listening operation of the acoustic unit 10. Once this trigger signal is communicated to the data processing module 100, the data processing module 100 may receive and process the sound signal via the unit 10 to generate raw body sound information (e.g., including measured values) or medical information based thereon. No body sound is acquired through the acoustic unit 10 when the pressure switch 40 is in the off-state (e.g., with the unit array 1020 in non-contact with the body).

As shown in FIG. 1, the unit array 1020 is coupled with the first coupling portion 1100. Since the first coupling portion 1100 is a relatively protruding portion, when the smart terminal 1 is moved in the body direction until it comes into contact with the body, the unit array 1020, in particular the electrode of the electrocardiogram unit 20, comes into contact relatively first. Then, the force that the user moves the smart terminal 1 in the body direction and/or the repulsive force that occurs when the unit array 1020 contacts the surface of the body may be switched to the on-state that applies pressure to the pressure switch 40, which in turn causes the pressure switch 40 to generate a trigger signal. When an electric signal according to switching is generated, a listening operation of the acoustic unit 10 is started.

In this way, when the acoustic unit 10 connected to the portion protruding from the body 1000 is brought into contact with the listening site (for example, the chest) of the target patient, the pressure switch is activated, so that the input mode of the smart terminal 1 is automatically changed to the auscultation mode and the electrocardiogram mode.

As a result, the smart terminal 1 automatically initiates a sound listening operation and an electrocardiogram measurement operation only by contacting the acoustic unit 10 with the body. The smart terminal 1 may not need to input a separate user command to an input device such as the operating unit 400 to initiate a sound listening operation or an electrocardiogram measurement operation.

The smart terminal 1 is configured to automatically perform, in real time, an operation of acquiring raw body information (i.e., body sound) on the auditory side and raw body information (that is, body electrocardiogram) on the electrocardiogram side, and processing the acquired raw body information to generate medical information of the subject, just by being used by the user for clinical treatment of the subject patient.

In addition, the user does not have to wear a short stethoscope on his ear and listen to the patient for stethoscope. That is, clinical treatment may be performed more easily.

In addition, the smart terminal 1 is configured to measure the body electrocardiogram more accurately or conveniently.

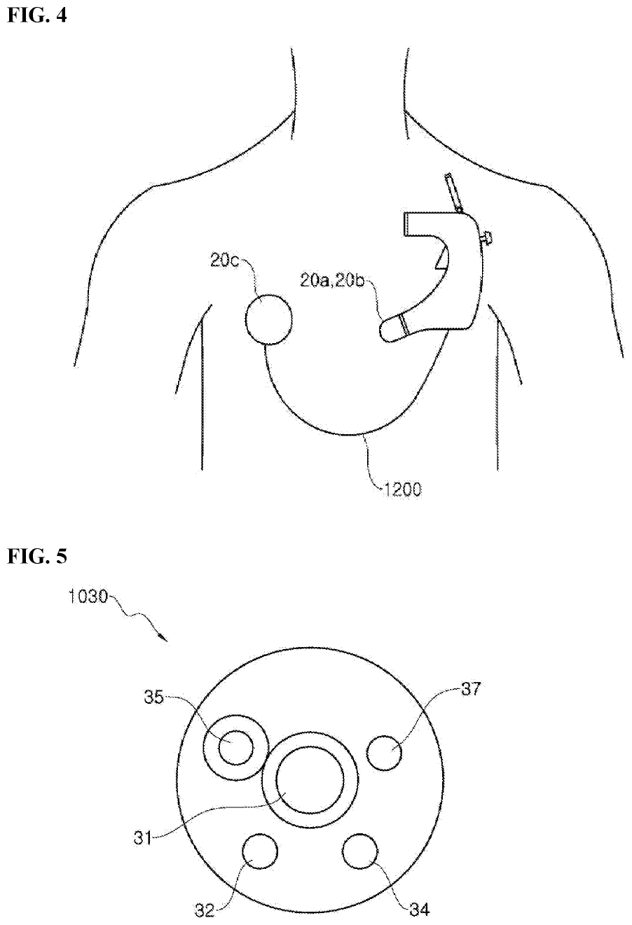

FIG. 4 illustrates a process of measuring an electrocardiogram of a subject through a plurality of electrocardiogram units, according to an embodiment of the present application.

Referring to FIG. 4, the plurality of electrocardiogram units 20 may include some electrodes (e.g., 20c) configured to extend from the body 1000.

The remaining electrodes 20c other than the electrodes 20a and 20b of the unit array 1020 may be exposed on the lower surface of the body 1000.

The partial electrodes 20c and the body 1000 are coupled to a connecting member 1200 configured to reduce or increase the relative distance therebetween. The connecting member may be a probe or a wire. On the other hand, the unit array 1020 in which the remaining electrocardiogram units 20a, 20b are installed is coupled to the body 1000 such that the relative distance from the body 1000 changes less than the change in relative distance from the electrode 20c and the body 1000. For example, the unit array 1020 may be coupled such that the relative distance from the body 1000 varies by a change in the pressure switch 40.

In one embodiment, the connecting member 1200 may be a retractable probe into the body 1000.

In another embodiment, the connecting member 1200 may be a probe that is rollable about a central axis coupled to the body 1000.

In another embodiment, the connecting member 1200 may include a part or all of a flexible material such that the connecting member 1200 has elasticity.

In another embodiment, the connecting member 1200 may include a foldable or rollable probe, and/or a combination of portions made of a flexible material.

By having such a connecting member 1200, the smart terminal 1 may minimize the volume or size of the smart terminal 1 while more easily contacting the partial electrode 20c with a particular portion other than the heart portion. In one embodiment, the plurality of electrocardiogram units 20a, 20b, 20c may initiate an electrocardiogram measurement operation when each electrode is in contact with the surface of the body, respectively. The plurality of electrocardiogram units 20a, 20b, 20c may contact a surface of a body to initiate an electrocardiogram measurement operation in response to receiving a weak electrical signal on the surface of the body.

In one embodiment, the smart terminal 1 may further respond to an electrical signal of the pressure switch 40 to cause the electrocardiogram unit 20 to initiate an electrocardiogram measurement operation. The smart terminal 1 may start the electrocardiogram measurement operation by applying pressure to the pressure switch 40 and switching to the on-state at the same time as the electrocardiogram units 20a and 20b of the unit array 1020 contact the skin. That is, the smart terminal 1 may start the electrocardiogram measurement operation when both the condition that the electrocardiogram signal flow opens and the condition that the pressure switch 40 is turned on are achieved. In this case, the electrical signal of the pressure switch 40 is a trigger signal for initiating an sound listening operation of the acoustic unit 10 and an electrocardiogram measurement operation of the electrocardiogram unit 20. When this trigger signal is communicated to the data processing module 100, the data processing module 10 may receive and process the signal via the units 10, 20 to generate raw body sound and raw body electrocardiogram information.

The smart terminal 1 is a single lead electrocardiogram, but it is very useful because it may immediately check the electrocardiogram during medical treatment. There is also the advantage of being able to extract various types of body electrocardiograms measured by adjusting the position at which each lead is brought into contact.

The body sound acquired by the acoustic unit 10 and/or the body electrocardiogram acquired by the electrocardiogram unit 20 are supplied to the data processing module 100 to generate medical information of a subject of the subject.

Referring again to FIG. 1, the photographing unit 30 is an input component that photographs a body to obtain a body image. In the alternative embodiments, the smart terminal 1 may acquire raw information of a visual aspect of a body part of a clinical subject by the photographing unit 30. The raw information of the visual aspect for this body part is used to generate visual medical information. The at least one photographing unit 30 includes at least one main photographing unit 31. In this case, the smart terminal 1 may include a white light source 32 and/or a fluorescent light source 34.

The main photographing unit 31 may be an input component that photographs an image in response to visible light and/or ultraviolet light. The main photographing unit 31 may be configured to photograph an image having a plurality of color channels (e.g., RGB channels). The main photographing unit 31 may be, for example, a camera, a digital camera, or a multispectral image sensor.

The white light source 32 is a light source that outputs light having a wavelength of at least a part of the visible light band. The white light source 32 may be, for example, a visible LED light lamp or other light source commonly used as illumination for camera photographing.

The fluorescent light source 34 is a light source that emits energy excited by excitation light into light. The fluorescent light source 34 is used as a fluorescent illumination for obtaining a fluorescent image. The fluorescent light source 34 may be, for example, but is not limited to, a UV output lamp.

When the light emitted from the white light source 32 and/or the fluorescent light source 34 is irradiated and reflected from the body, the main photographing unit 31 generates a general body image or a fluorescent body image in response to the reflected light. Then, the smart terminal 1 may obtain a general body image or a fluorescent body image.

Here, the main photographing unit, the white light source 32 and the fluorescent light source 34 may be implemented or realized by one or more processors included in a smart terminal 1.

The main photographing unit 31 and the light sources 32 and 34 may be coupled to the second coupling portion 1300.

In some embodiments, the at least one photographing unit 30 may further include an infrared unit 35 and/or an illuminance sensor 37. The infrared unit 35 is a component that generates an infrared image in response to reflected light in the infrared band. The infrared unit 35 may include, for example, an infrared camera and/or an infrared sensor.

Here, the infrared unit 35 and an illuminance sensor 37 may be implemented or realized by one or more processors included in a smart terminal 1.

The smart terminal 1 may obtain a two-dimensional image of a single color channel, such as an infrared image according to an infrared intensity, by the infrared camera 35.

When the smart terminal 1 includes the infrared unit 35, a temperature image of the patient may be generated for the photographing area. When inflammation occurs in the body, the inflammatory part has a relatively high temperature compared to the non-inflammatory part. The temperature image of the patient may also be used to determine whether the application is prone to inflammation of a particular site in the patient's body.

The illuminance sensor 37 is an input component configured to measure an illuminance value around the smart terminal 1 or detect a change in illuminance. Such an illuminance sensor 37 may be coupled with the second coupling portion 1300.

The illuminance sensor 37 may collect an illuminance value of a subject photographed by the main photographing unit 30 or an illuminant value around the second coupling portion 1300. The illuminance sensor 37 may be, for example, but is not limited to, an Ambient light sensor.

The photographing units 31, 35, 37 and the light sources 32, 34 may form a unit array 1030 separately from the unit array 1020.

FIG. 5 is a diagram illustrating a unit array including a plurality of photographing units, according to an embodiment of the present application.

Referring to FIG. 5, the photographing units 31, 35, 37 and the light sources 32, 34 are arranged on the board 1031 to form the unit array 1030.

In one embodiment, the remaining photographing units 35, 37 and/or the light sources 32, 34 may be arranged around the main photographing unit 31 in the unit array 1030. For example, the main photographing unit 31 may be located in the center of the unit array 1030 and the remaining components 32, 34, 35, 37 may be arranged in a circular shape along the main photographing unit 3. The unit array 1030 is coupled to a second coupling portion 1300 that protrudes relatively from the body 1000.

In this way, the photographing unit 30 has a relatively protruding structure, so that the smart terminal 1 may be inserted at least partially into a body internal structure, such as in the mouth, so that the body internal structure may be photographed relatively easily.

The body image obtained by the photographing unit 30 is supplied to the data processing module 100 to generate medical information.

The smart terminal 1 acquires raw body information including body sound and body electrocardiogram by the acoustic unit 10, the electrocardiogram unit 20, and the photographing unit 30. The acquired raw body information is supplied to the data processing module 100.

The data processing module 100 includes at least one processor, and controls overall driving and operation of the smart terminal 1 for generating medical information. The data processing module 100 may activate or switch a mode for acquiring raw body information.

In addition, the data processing module 100 may operate as a measurement module that processes an electrocardiogram signal and an sound signal to measure an electrocardiogram and an sound value. Such measurement results may also be included in the raw body information.

In addition, the data processing module 100 may generate medical information of the subject based on the raw body information.

The data processing module 100 includes: a data processing unit 110; and a data transmission/reception unit 150.

Here, the data processing unit 110; and the data transmission/reception unit 150 may be implemented or realized by one or more processors included in a smart terminal 1.

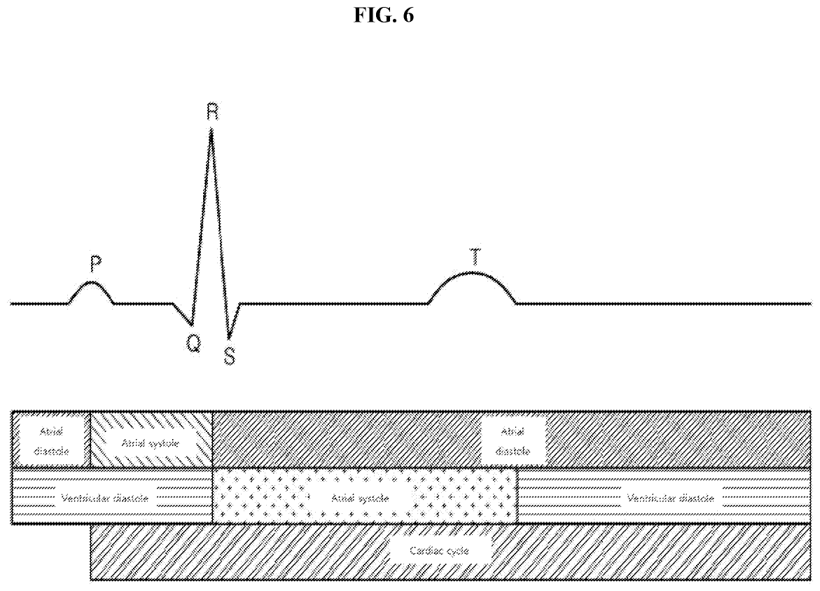

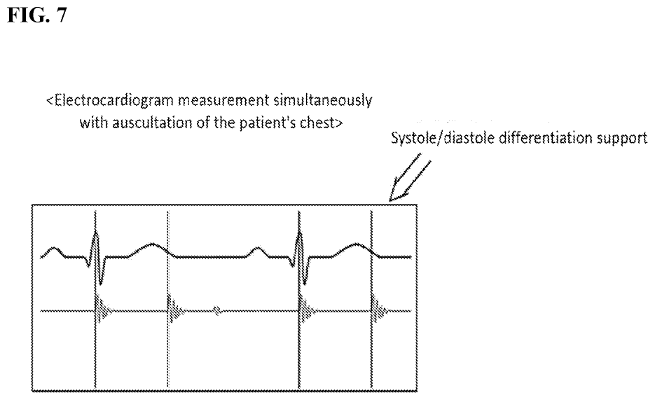

The data processing unit 110 processes the raw body information to generate medical information of the subject. The raw body information includes body sound and/or body electrocardiogram. In addition, the raw body information may further include a photographed body image. The medical information generated by the data processing unit 110 will be described in more detail with reference to FIGS. 6 and 7 below.

On the other hand, the data transmission/reception unit 150 may communicate with an external device by various communication methods capable of networking an object and the object. The communication method may include: wired/wireless communication, 3G, 4G, 5G wired/wireous Internet, and the like. For example, the communication method includes, but is not limited to: the Internet such as the World Wide Web (WWW), a network such as an intranet and/or a cellular telephone network, a wireless network, and a wireless communication standard using a communication protocol including a Global System for Mobile Network (GSM), Code Division Multiple Access (CDMA), Wideband Code Division Multiple Access (“W-CDMA”), Time Division Multiple Access (“TDMA”), Bluetooth, Wi-Fi, and long term evolution (“LTE”) scheme.

The data processing module 100 may transmit the raw body information received by the data processing unit 110 or the generated medical information of the subject to the external device through the data transmission/reception unit 150.

Referring again to FIG. 1, the data processing unit 110 may independently process the listened body sound or the measured body electrocardiogram to generate medical information of the subject based on a single aspect.

Alternatively, when the data processing unit 110 acquires raw body information of various aspects including an auditory aspect, an electrocardiographic aspect, and/or a visual aspect, the data processing module 100 may generate medical information of a subject based on the plurality of aspects. The medical information of the subject based on the plurality of aspects may be implemented as fusion data generated by integrating the raw body information of the plurality of aspects in a single format.

In certain embodiments, the data processing unit 110 processes the raw body information in a preset data form to generate medical information of the subject. Here, the preset data format is a data format acceptable for an application program provided in an external device that communicates with the data transmission/reception unit 150. Data having this format is directly available in an installed application when transmitted to the external device.

The application program of the external device may be an application program for clinical practice. The application program includes, for example, an artificial intelligence algorithm for clinical practice, such as an artificial intelligence algorithm that predicts a patient's disease name.

The external device provided with the application program may be a personal computer provided with an electronic medical record or a server provided with an artificial intelligence algorithm for clinical practice.

First, an operation of the data processing unit 110 will be described in an exemplary case in which the smart terminal 1 receives sound information.

In one example, raw body sound such as lung sound or deep sound is input to the data processing unit 110 through a acoustic unit 30. The data processing unit 110 extracts a one-dimensional sound vector x of a preset length N(=frequency (e.g., Hz)*time (e.g., seconds)) from the input raw body sound.

In addition, the data processing unit 110 may normalize the one-dimensional sound vector x. The normalization may be, for example, but not limited to, a min-max scaling scheme, a z-transformation, or the like.

The data processing unit 110 may be configured to generate the medical information of the subject by using the one-dimensional sound vector x itself, and/or to convert the one-dimensional sound vector x into an image format to generate the medical data of the subject. The medical information of the foregoing subject may be based only on sound information. On the other hand, the medical information of the subject described below may be based on sound information as well as other aspects of raw body information.

In one embodiment, the data processing unit 110 may convert the one-dimensional sound vector x into a spectrum format, a power spectrum format, or a spectrum format. Image data in a spectral format, a power spectral format, or a spectrogram format is generated based on a local Fourier transform (STFT). For example, a spectral format, a power spectral format, or a spectrogram format is represented by the following equation.

$$Spectrum = STFT ( x ) \qquad [ Equation 1 ]$$

$$Power\_Spectrum = {\vert STFT ( x ) \vert}^{2} \qquad [ Equation 2 ]$$

$$Spectrogram = log ( {\vert STFT ( x ) \vert}^{2} ) \qquad [ Equation 3 ]$$

The one-dimensional sound vector x is converted into sound data in an image format, represented by a C×M matrix, through Equations 1, 2, or 3 described above. where C represents the channel of the image (e.g., the number of channels of the channel axis in the spectrum), and M represents the time of the image (i.e., the length of the time axis in the spectrum).

In addition, the data processing unit 110 may normalize sound data in a converted image format. The normalization may be performed by, for example, but not limited to, a min-max scaling method, a z-transformation method, or the like.

The data processing unit 110 may perform normalization over a part or the entire range of the one-dimensional sound vector x in the process of generating sound data in the C×M format.

In one embodiment, the data processing unit 110 may apply the normalization processing over the entire range. The full range here applies for both the channel axis and the time axis.

In another embodiment, the data processing unit 110 may apply the normalization processing over some range. Here, some ranges are applied channel-wise.

Next, the operation of the data processing unit 110 will be described in an exemplary case in which the smart terminal 1 receives an input of a body electrocardiogram.

In one example, the data processing unit 110 may process an input raw electrocardiogram signal to extract electrocardiogram measurement data from the raw electrocardiogram signals. Since a raw electrocardiogram signal is input through the unit array 1020 of FIG. 2, the raw electrocardiogram is simultaneously measured in a time range in which the one-dimensional sound vector x is extracted, and is data synchronized with each other.

The electrocardiogram data may be expressed in an L×T matrix format. where L represents the electrocardiogram channel and depends on the number of leads. For example, L may have a value of any one of 1 to 12. T represents a time (e.g., a number of time axes), and may be variable according to a sampling rate.

In addition, the data processing unit 110 may normalize the electrocardiogram data for each channel. The normalization may be performed, for example, through min-max scaling method, z-transformation, etc., but is not limited thereto.

The data processing unit 110 may generate medical information of the subject using the electrocardiogram data itself in the L×T matrix format (normalized or not), and/or may process the electrocardiogram information to generate medical information of a subject based on a plurality of aspects of raw body information. The medical information of the foregoing subject may be based only on electrocardiogram information. On the other hand, the medical information of the subject described below may be based on electrocardiogram information as well as other aspects of raw body information, such as based on body electrocardiogram and body sound. The data processing unit 110 processes the electrocardiogram data for each of the electrocardiogram channels L in order to reinforce the sound data in the form of a silver C×M matrix.

In one embodiment, the data processing unit 110 may generate medical information of a subject who synchronizes the body electrocardiogram and the body sound on a time domain. In this synchronization process, the sound data is reinforced by the electrocardiogram data.

The data processing unit 110 may be configured to process the electrocardiogram data in the form of an L×T matrix to generate medical information of a subject who partitions an sound time interval (e.g., a stethoscope time interval) in order to reinforce the sound data. The medical information of this subject is based on body sound and body electrocardiogram.

The data processing unit 110 may generate medical information of the subject by converting to have a matrix format matching a matrix size of the sound data in an L×T matrix format. For example, the transformed electrocardiogram data may have a C×M matrix format.

The data processing module 100 may include at least one artificial neural network learned in advance. Each artificial neural network is configured to receive data of an input channel matching the electrocardiogram channel L, and is learned to calculate a one-channel vector for classifying a specific sound segment.

FIG. 6 illustrates an sound segment partitioned using a measured body electrocardiogram, according to an embodiment of the present application.

Referring to FIG. 6, the particular sound period may include one or more sub-periods of a cardiac cycle. The smart terminal 1 may include a plurality of artificial neural networks corresponding to each segment to classify a plurality of sound segments. For example, the smart terminal 1 may include a plurality of artificial neural networks for classifying an atrial diastole, an atrial systole, an atrial diastole and/or a ventricular diastole.

Each artificial neural network is configured to calculate output data consisting of a first label value at a time point corresponding to its specific sound segment learned to classify in the input data, and a second label value at a remaining time point. In one example, the particular sound segment may be an atrial systolic segment. The output vector of the artificial neural network for classifying the atrial systole may then be a vector having a value of 1 at the time point of the atrial systole and 0 at the remaining time point.

The data processing unit 110 may generate electrocardiogram data in a C×M matrix format by resizing a one-channel vector of the artificial neural network to a value equal to an M axis in a matrix of the sound data, and replicating the adjusted one-channel vector by a value of C in the matrix of the sound information, and stack the electrocardiogram data converted into the C×M matrix form into the sound data in the C×M process format, so as to generate medical information of a subject based on body electrocardiogram and body sound.

The sound data in the C×M matrix format and the electrocardiogram data in the C×M matrix format only match the matrix structure, and the data components in each matrix are separate from each other.

The segment information of the body sound in the generated medical information of the subject may be associated with a body electrocardiogram (e.g., a signal measurement value).

The data processing unit 110 may stack electrocardiogram data on or below sound data.

The data processing unit 110 may stack electrocardiogram data for each section. For example, when per-segment electrocardiogram data is generated for Z particular sound segments, the Z electrocardiogram data may be stacked up or down relative to the sound data. Then, the data processing unit 110 may generate medical information of the subject in the form of C×M×(Z+1).

In this way, the data processing module 100 may generate medical information of a subject, which is acquired in multiple input modes and is composed of multi-modal input data.

FIG. 7 shows a result of synchronizing body electrocardiogram and body sound based on partitioning of the heart cycle, according to an embodiment of the present application.

Referring to FIG. 7, medical information based on the body electrocardiogram and body sound synchronized on the time domain may be generated in the smart terminal 1. When such medical information is transmitted to the external device and utilized as training data, the artificial intelligence performance of an application program including an artificial intelligence algorithm may be improved.

For example, assume that the application program of the external device is programmed to distinguish between systolic or diastolic groups in body electrocardiogram using an artificial intelligence algorithm. The data transmission/reception unit 150 transmits the medical information of the subject including the synchronized information to the external device. Synchronized information in the subject's medical information allows the application to more accurately differentiate the patient's systolic or diastolic phase compared to non-synchronized information (e.g., such as body electrocardiogram alone).

In the alternative embodiments, the data processing unit 110 may generate medical information of the subject based on the sound information, electrocardiogram information, and photographing information.

When the body image is photographed through the unit array 1030 of FIG. 5, the raw body photographing information includes image data of the main photographing unit 31; image data of the infrared unit 35; and image data of the illuminance sensor 37. Further, the raw body photographing information may include driving data of the fluorescent light source 34.

The image data of the main photographing unit 31 may be three-channel data, such as an RGB channel. The image data of the infrared unit 35 may be one-channel data. The illuminance sensor 37 may be three-channel data.

The driving data of the fluorescent light source 34 indicates the presence or absence of an output of a fluorescent light source 34 (e.g., a UV lamp), and may be one-channel data.

The data processing unit 110 may generate medical information based on two-dimensional image input of up to 8 channels. The medical information may have a form of input data of an artificial intelligence algorithm of an external device. For example, the input data form may be 3-tensor data.

In the above embodiment, the data processing unit 110 may process eight channels of two-dimensional image input, so that a channel of the illuminance sensor 37 in the eight channels broadcasts a scalar value indicating a measured value of the illumination sensor 37 into a w×h matrix. Here, w and h may be a data width and a data height in a form of input data of an artificial intelligence algorithm of an external device. The measured value of the illuminance sensor 37 may correspond to three scalar values.

In addition, the data processing unit 110 may define the driving data of the light source 34 as a binary scalar value (e.g., on is 1, off is 0). Then, the data processing unit 110 may process the two-dimensional image input of the eight channels, and broadcast a scalar value indicating whether the light source 34 is driven in the eight channels in the two-dimensional video input to a w×h matrix.

The data processing unit 110 converts an 8-channel two-dimensional image input to construct 3-tensor data.

In addition, the data processing unit 110 may perform normalization processing on the transformed image input.

In the medical information, a photographing time of photographing information (e.g., a photographing image) may be synchronized with an electrocardiogram time or an sound time.

In this way, when the medical information of the subject having the preset data form is generated from the body sound, the body electrocardiogram, and the body image by the data processing unit 110, the data transmission/reception unit 150 transmits the medical information of a subject in the above format to the external device. Upon receiving the medical information of the subject, the external device applies the medical information of the subject to an application program for the clinical treatment to perform a clinical treatment operation on a subject patient who has provided the medical information of a subject.

Because the medical information of the subject is based on various input signals acquired through various types of sensors, the performance of the artificial intelligence algorithm analysis of the application is improved. Application of fusion data based on body sound of a lung sound/lung ultrasonic sensor to an application program improves the performance of artificial intelligence algorithm analysis of the application program.

In addition, when the body sound and the body electrocardiogram are synchronized in the time domain and transmitted to the external device together, the artificial intelligence algorithm analysis performance becomes more accurate.

In addition, the auxiliary sound unit 50 is a component that listens to a sound different from a body sound to be used in clinical practice. The auxiliary sound unit 50 may listen to non-body sounds, such as conversations that occur during clinical practice.

The auxiliary sound unit 50 may be, for example, a microphone. The auxiliary sound unit 50 may have a different listening specification than the acoustic unit 10.

The auxiliary sensor 60 is a sensor that assists in photographing the subject more accurately and without shaking to obtain a sharper body image.

The auxiliary sensor 60 may comprise an acceleration sensor and/or a rotation sensor. The smart terminal 1 acquires the motion information of the smart terminal 1 based on the acceleration information acquired by the acceleration sensor. The motion information includes a motion direction and/or a motion intensity.

The smart terminal 1 acquires the rotation information of the smart terminal 1 based on the rotation information acquired by the rotation sensor. The rotation information includes a rotation direction and/or a rotation intensity.

When the smart terminal 1 includes the auxiliary sensor 60, the image stabilization may be performed by using the detection result of the auxiliary sensor 60. The data processing module 100 may generate a body image in which a captured image is aligned and corrected by using motion information and/or rotation information. In addition, the data processing module 100 may generate medical information of a subject who has processed motion information and/or rotation information in the preset data form. In addition to the processing result of the raw body information, it is also possible to supply the medical information of the subject, including the information on the direction in which the smart terminal 1 moves when moving the photographing unit 30 to the specific affected part of the patient, to the external device. As a result, the medical information of the subject provides additional information for analyzing where the application program of the external device moves the smart terminal 1.

The display unit 200 displays information for interacting with a user for the purpose of generating medical information, or displays generated medical information.

The display unit 200 may display an operation description/guide for generating medical information of the smart terminal 1. In addition, the display unit 200 may display an instruction for manipulating the photographing position of the to-be-inputted body image, and an instruction for maneuvering the focal position of the to to-be-inputted body image.

In addition, the display unit 200 displays a visual result of the input raw body information (e.g., body electrocardiogram). The data processing unit 110 may visualize body sound (e.g., auscultation sound or ultrasound) in a graph form and display it in the display unit 200. The data processing unit 110 may visualize and display body electrocardiogram or impedance information used for measurement of the body electrocardiogram in the display unit 200.

In addition, the display unit 200 displays medical information generated in the data processing unit 110. The display unit 200 may display a result of analyzing the raw body information.

The sound output unit 300 is a component that receives and converts an electrical signal into an sound signal, and includes a speaker or the like.

The sound output unit 300 may output a body sound such as a stethoscope sound or a Doppler sound.

When the extensible electrode 20c, which is coupled to the lower end of the smart terminal, is in contact with the chest of the patient to perform auscultation, a beep sound that distinguishes between the systolic and the diastolic may be output to the sound output unit 300 and/or information visualizing the beep sound may be output to the display unit 200.

The sound output unit 300 may output a device-operated sound indicating performance of a specific operation for each operation.

The sound output unit 300 may output an instruction for inducing acquiring raw body information. For example, the sound output unit 300 may output a sound of “inhale”.

The operation unit 400 receives the information required for generating medical information, or acquires a user instruction for controlling a process required for generating the medical information.

The operating unit 400 may further include a first operating unit 420 and/or a second operating unit 430.

Any one of the first operating unit 420 and the second operating unit 430 inputs a trigger instruction that initiates a specific operation. For example, the first operating unit 420 may input a trigger instruction.

The first operating unit 420 and the second operating unit 430 are provided in directions opposite to each other in the body 1000. For example, the first operating unit 420 is installed in a body 1000 part in a direction in which the smart terminal 1 faces the body of the target, that is, in a front part of the body 1000. The second operating unit 430 may be provided in a portion of the body 1000 in a direction opposite to the front operating unit 420, that is, in a rear portion of the body 1,000.

The operating units 420 and 430 may be implemented in the form of buttons, dials, joysticks, and switches. For example, the first operating unit 420 may be implemented in the form of a switch that turns on the trigger function. The second operating unit 430 may be implemented in the form of a stick or a button.

The smart terminal 1 may select a light source by the first operating unit 420 or the second operating unit 430, adjust the intensity of the selected light source, adjust the focus, or initiate a photographing operation. The user may manually adjust the photographing focus and the exposure time through the operating unit 400.

In addition, the smart terminal 1 may adjust the intensity of the output sound by the first operating unit 420 or the second operating unit 430.

In one embodiment, in the smart terminal 1, the operating unit 400 may initiate an operation of mixing sounds of different frequency bands among body sounds by the first operating unit 420 or the second operating unit 430, or may adjust a mixing ratio.

The smart terminal 1 may select the type of the body image by the operating unit 400. For example, the operating unit 400 may input a photographing mode instruction for selecting a body image type to generate a visible light or infrared body image. Such an photographing mode may include a visible light photographing mode; an infrared photographing mode; and an optical fiber photographing mode.

In addition, the operating unit 400 may input a command for activating the white light source 32 or the fluorescent light source 34 in the visible light photographing mode. By means of the light source selection instruction, the smart terminal 1 may assist in the clinical practice of the ophthalmology or dermatology.

In addition, the operating unit 400 may be further configured to input a recording instruction for recording a listened body sound.

In embodiments according to another aspect of the present application, the smart terminal 1 may include components related to an electrocardiogram signal among the components of FIGS. 1 to 5. In the embodiments, the smart terminal 1 may include the remaining units 10, 30, 40, 60, 100, 110, 150, 200, 300, 420, 430, 1100, 1200, 1300, excluding the electrocardiogram unit 20 and its related components (e.g., electrodes 20a-20c).

Specifically, the smart terminal 1 according to another aspect of the present application may include at least one acoustic unit 10 connected to a body of the smart terminal 1, and configured to acquire a body sound of a subject; a data processing module 100 configured to process raw body information of at least one of the acquired body sound and a body electrocardiogram in a preset data form to generate medical information of the subject; and a pressure switch 40 configured to generate an electrical signal when a pressure is applied and switched.

In one embodiment, the smart terminal 1 may initiate a listening operation by the acoustic unit in response to an electrical signal of the pressure switch 40 being generated.

In one embodiment, the interfaces of the at least one acoustic unit 10 may be arranged on the same plane to form a unit array 1020. The unit array 1020 may be connected to a first coupling portion of the body, and the first coupling portion may protrude from a surface of another portion of the body.

In one embodiment, the pressure switch 40 may be disposed between the first coupling portion and the unit array. Pressure may be applied to the pressure switch 40 to generate the electrical signal as the protruding unit array 1020 contacts a surface of a body part that will acquire body sound. The operation of the pressure switch 40 and the structure of the unit array 1020 have been described above, and detailed description thereof will be omitted.

In one embodiment, the smart terminal 1 may further include a 2D cross-sectional ultrasonic sensor or an ultrasonic sensor 16 using a Doppler effect.

In one embodiment, the unit array 1020 may include a plurality of acoustic units 21, 23 for listening to body sound of different frequency bands. The plurality of acoustic units 21, 23 may include a first acoustic unit 21 for listening to relatively high-pitched body sound in an audible frequency band and a second acoustic unit 23 for listening to relatively low-pitched body sounds in the audible frequency bands.

In one embodiment, the smart terminal 1 may further include an operation unit that mixes the listened high-pitched sound and low-pitched sound, or controls a mixing ratio.

In one embodiment, the smart terminal 1 may further include one or more of: an sound output unit 300 configured to output an acquired body sound as an sound signal; and a display unit 200 configured to display raw body information including the acquired body sound, or medical information of a subject.

In one embodiment, the smart terminal 1 may further include a data transmission/reception unit 150 configured to transmit medical information of the subject to an external device equipped with a diagnostic program or an artificial intelligence program.

In one embodiment, the data processing unit 110 of the data processing module 100 may be configured to process the body sound in a preset data form to generate medical information of the subject. The preset data format may be a data format acceptable for an application program installed in an external device that communicates with the data transmission/reception unit.

In one embodiment, the data processing unit 110 may be configured to extract the one-dimensional sound vector x of the preset length N from the body sound, and generate medical information of the subject by using the one-dimensional sound vector x itself, or convert the one-dimensional sound vector x into an image format to generate medical information of a subject.

In one embodiment, the data processing unit 110 may be configured to convert the one-dimensional sound vector x into a spectrum format, a power spectrum format, or a spectrum format to generate sound data in an image format represented by a C×M matrix. The medical information of the subject includes sound data in the image format, wherein C represents a channel of body sound, and M represents a time of body sound.

In one embodiment, the smart terminal 1 is a smart terminal that further acquires medical information of a visual aspect, and may further include at least one photographing unit 30 that photographs a body. The data processing module 100 may process the body image obtained by the at least one photographing unit 30 in a preset data form, so that the medical information of the subject further includes a processing result of the body image.

In one embodiment, the smart terminal 1 may further include an operation unit 400 that selects the photographing unit 30 or controls specifications of the photographing unit 30.

In one embodiment, the at least one photographing unit may include one or more of: at least one visible light photographing unit 31; at least one infrared photographing unit 35; and at least one illuminance sensor 37.

In one embodiment, when the smart terminal 1 includes at least one visible light photographing unit 31, the smart terminal 1 may further include one or more of a white light source 32 and a fluorescent light source 34.

In one embodiment, when the smart terminal 1 includes at least one infrared photographing unit 35, the data processing module 100 may be further configured to generate a temperature image of an photographing area for checking whether an inflammation reaction of a specific site occurs.

These components and their operations have been described above, and detailed description thereof is omitted.

It will be apparent to those skilled in the art that the smart terminal 1 may include other components not described herein. For example, the smart terminal 1 may include a network interface, an input device for a data entry, a memory for storing data, and other hardware elements necessary for the operations described herein, including an output device for display, printing, or other data presentation.

The operation by the smart terminal 1 according to the embodiments described above may be at least partially implemented as a computer program and recorded on a computer-readable recording medium. For example, implemented with a program product comprised of a computer-readable medium comprising program code, which may be executed by a processor to perform any or all of the steps, operations, or processes described.

The computer-readable recording medium includes all kinds of recording identification devices in which data that may be read by a computer is stored. Examples of the computer-readable recording medium include a ROM, a RAM, a CD-ROM, a magnetic tape, a floppy disk, an optical data storage identification device, and the like. In addition, the computer-readable recording medium may be distributed over a networked computer system, so that the computer-readable code is stored and executed in a distributed manner. In addition, functional programs, codes, and code segments for implementing the present embodiment may be easily understood by those skilled in the art to which the present embodiment belongs.

While the invention described above has been described with reference to the embodiments shown in the drawings, it will be understood by those skilled in the art that these are merely exemplary and that various modifications and variations of the embodiments are possible therefrom. However, such modifications should be considered to be within the technical protection scope of the present invention. Therefore, the true technical protection scope of the present invention should be determined by the technical idea of the appended claims.

### INDUSTRIAL EXPLOITATION

A smart terminal that acquires medical information including an auditory aspect according to one embodiment may be used in a clinical treatment industry that utilizes medical information by acquiring medical information in real time in a format usable for clinical treatment.

What is claimed is:

1. A smart terminal for acquiring medical information including an auditory aspect, comprising: one or more processors and a memory for storing instructions executed by the one or more processors,
wherein the instructions, when executed by the one or more processors, allow the one or more processors to:
cause at least one acoustic unit connected to the body of the smart terminal to acquire body sound of a subject,
and cause a data processing module included in the smart terminal to process at least one raw body information among the acquired body sound and body electrocardiogram into a preset data format to generate medical information of the subject,
and a pressure switch included in the smart terminal generates an electric signal when pressure is applied and and switched.

2. The smart terminal according to claim 1,
wherein the instructions, when executed by the one or more processors, allow the one or more processors to:
cause the acoustic unit to initiate a listening operation in response to the electric signal of the pressure switch being generated.

3. The smart terminal according to claim 1,
wherein the interfaces of at least one acoustic unit are arranged on the same plane to form a unit array, and
the unit array is connected to a first coupling portion of the body, and the first coupling portion protrudes more than a surface of another portion of the body.

4. The smart terminal according to claim 3,
wherein the pressure switch is arranged between the first coupling portion and the unit array,
the instructions, when executed by the one or more processors, allow the one or more processors to:
apply pressure to the pressure switch and generate the electrical signal according to the contact of the protruding unit array with the surface of the body part from which body sound is to be acquired.

5. The smart terminal according to claim 1,
further comprising: a 2D cross-sectional ultrasonic sensor or an ultrasonic sensor using the Doppler effect.

6. The smart terminal according to claim 3,
wherein the instructions, when executed by the one or more processors, allow the one or more processors to:
cause the plurality of acoustic units included in the unit array to listen to body sound of different frequency bands,
cause a first acoustic unit included in the plurality of acoustic units to listen to relatively high-pitched body sound in audible frequency band,
and cause a second acoustic unit included in the plurality of acoustic units to listen to relatively low-pitched body sound in the audible frequency band.

7. The smart terminal according to claim 6,
wherein the instructions, when executed by the one or more processors, allow the one or more processors to:
cause an operating unit included in the smart terminal to mix the heard high-pitched sound or low-pitched sound, or control the mixing ratio.

8. The smart terminal according to claim 1,
wherein the instructions, when executed by the one or more processors, allow the one or more processors to:
cause a sound output unit included in the smart terminal to output the acquired body sound as an sound signal, and
cause a display unit included in the smart terminal to display raw body information including body sound acquired, or medical information of a subject.

9. The smart terminal according to claim 1,
wherein the instructions, when executed by the one or more processors, allow the one or more processors to:
cause a data transmission/reception unit included in the smart terminal to transmit the medical information of the subject to an external device having a diagnostic program or an artificial intelligence program installed.

10. The smart terminal according to claim 9,
wherein the instructions, when executed by the one or more processors, allow the one or more processors to:
cause the data processing module to process the body sound signal into a preset data format to generate medical information of the subject, and
the preset data format is a data format acceptable to an application program installed in an external device communicating with the data transmission/reception unit.

11. The smart terminal according to claim 9,
wherein the instructions, when executed by the one or more processors, allow the one or more processors to:
cause the data processing module to extract a one-dimensional sound vector x of a preset length N from the body sound, and generate medical information of the subject using the one-dimensional sound vector x itself, or generate medical information of the subject by converting the one-dimensional sound vector x into an image format.

12. The smart terminal according to claim 11,
wherein the instructions, when executed by the one or more processors, allow the one or more processors to:
cause the data processing module to convert the one-dimensional sound vector x into a spectrum format, a power spectrum format, or a spectrogram format to generate sound data in an image format, expressed as a C×M matrix,
the medical information of the subject includes the sound data in the image format, and
the C represents a channel of body sound, and the M represents time of body sound.

13. The smart terminal according to claim 1,
wherein the instructions, when executed by the one or more processors, allow the one or more processors to:
cause at least one photographing unit included in the smart terminal to photograph the body to acquire more medical information of visual aspect, and
cause the data processing module to process the body image obtained by the at least one photographing unit into a preset data format so that the medical information of the subject further includes a result of processing the body image.

14. The smart terminal according to claim 13,
wherein the instructions, when executed by the one or more processors, allow the one or more processors to:
cause the operating unit included in the smart terminal to select the photographing unit or control the specifications of the photographing unit.

15. The smart terminal according to claim 13,
wherein the at least one photographing unit includes one or more of at least one visible light photographing unit; at least one infrared photographing unit; and at least one illuminance sensor.

16. The smart terminal according to claim 15,
wherein the smart terminal further includes one or more of a white light source and a fluorescent light source when the smart terminal includes at least one visible light photographing unit.

17. The smart terminal according to claim 15,
wherein when the smart terminal includes at least one infrared photographing unit, the instructions, when executed by the one or more processors, allow the one or more processors to:
cause the data processing module to generate a temperature image of a photographed area to determine whether there is an inflammatory reaction in a specific area.
